(19) United States
(12) Patent Application Publication
(10) Pub. No.: US 2022/0249213 A1
(43) Pub. Date: Aug. 11, 2022

(54) TOOTH CLEANING DEVICE

(71) Applicant: SHENZHENSHI XIFUJI INTELLIGENT TECHNOLOGY CO., LTD., Shenzhen (CN)

(72) Inventors: Jiangnan He, Shenzhen (CN); Wei Huang, Shenzhen (CN); Zhiqiang Zeng, Shenzhen (CN); Yanhui Yang, Shenzhen (CN)

(21) Appl. No.: 17/168,179

(22) Filed: Feb. 5, 2021

(51) Int. Cl. A61C 17/20; A61B 1/24; A61B 1/00

(52) U.S. Cl. CPC A61C 17/20; A61B 1/00034; A61B 1/00045; A61B 1/24

(57) ABSTRACT

A tooth cleaning device includes a main body, a cleaning head extending out of the main body, and a camera unit. An upper end of the main body is provided with an inclined portion that is inclined downward and rearward. The camera unit is mounted to the inclined portion. An ultrasonic transducer is mounted to the inclined portion. The cleaning head is connected to the ultrasonic transducer. The ultrasonic transducer is rotatably connected to the main body.

### BACKGROUND OF THE INVENTION

### 1. Field of the Invention

The present invention relates to a tooth cleaning device, and more particularly to a tooth cleaning device that can be finely adjusted.

### 2. Description of the Prior Art

With the improvement of living standards, people pay more and more attention to oral hygiene. Cleaning teeth has become a common health care for people to maintain oral hygiene. A tooth cleaning device is a special apparatus for cleaning teeth, which is used to remove tartar, stains, plaque, etc.

A conventional tooth cleaning device with camera function generally includes a main body, a camera and a cleaning head. The orientation of the conventional cleaning head is generally immovable. It is difficult to adjust its orientation, which will affect the normal use of the driving head.

Accordingly, the inventor of the present invention has devoted himself based on his many years of practical experiences to solve these problems.

### SUMMARY OF THE INVENTION

In view of the defects of the prior art, the primary object of the present invention is to provide a tooth cleaning device that can be finely adjusted. It is more convenient to use. The orientation of a cleaning head can be finely adjusted for the working position of the cleaning head, so as to ensure the normal use of the cleaning head. Furthermore, during the rotation of the cleaning head, it is possible to prevent the camera unit from being damaged, so that the camera unit is well protected.

In order to achieve the above object, the present invention adopts the following technical solutions:

A tooth cleaning device comprises a main body, a cleaning head extending out of the main body, and a camera unit. An upper end of the main body is provided with an inclined portion that is inclined downward and rearward. The camera unit is mounted to the inclined portion. An ultrasonic transducer is mounted to the inclined portion. The cleaning head is connected to the ultrasonic transducer. The ultrasonic transducer is rotatably connected to the main body. The camera unit is connected to the inclined portion and adjustable to avoid the cleaning head when rotating.

Preferably, the ultrasonic transducer is provided with a rotation limiting portion for limiting a rotation angle of the ultrasonic transducer.

Preferably, the rotation limiting portion protrudes from an upper end face of the ultrasonic transducer, a locking protrusion is provided on the inclined portion, and the rotation limiting portion abuts against the locking protrusion.

Preferably, the ultrasonic transducer includes a mounting seat, a cylindrical portion mounted to the mounting seat, and a driving portion integrally connected to a top end of the cylindrical portion. The driving portion is detachably connected to the cleaning head. The rotation limiting portion is disposed on the mounting seat and/or the cylindrical portion.

Preferably, a function button is provided on the main body.

Preferably, a display screen is provided on the main body.

Preferably, the camera unit is rotatably connected to the main body to be turned back and forth.

Preferably, a front end face of the camera unit is formed with a first finger recess corresponding to the curvature of a user's finger surface.

Preferably, a front end face of the inclined portion is formed with a second finger recess corresponding to the curvature of a user's finger surface.

Preferably, a power source for power supply is provided in the main body, a charging port is provided on a lower end face of the main body, and the charging port is electrically connected to the power source.

Compared with the prior art, the present invention has obvious advantages and beneficial effects. Specifically, it is more convenient to use through the inclined portion. In particular, through the design of the ultrasonic transducer that is rotatable relative to the main body, the orientation of the cleaning head can be finely adjusted for the working position of the cleaning head, so as to ensure the normal use of the cleaning head. Furthermore, through the adjustable camera unit, during the rotation of the cleaning head, it is possible to prevent the camera unit from being damaged, so that the camera unit is well protected.

Secondly, the rotation limiting portion is configured to limit the rotation angle of the ultrasonic transducer, so as prevent the internal wires from breaking due to the excessive rotation of the ultrasonic transducer and to prolong the service life of the product.

Furthermore, through the cooperation of the first finger recess and the second finger recess, it is possible to avoid slippage and shifting when the camera unit is used and turned, thereby improving the anti-slip effect.

In addition, the design of the overall structure is ingenious and reasonable, the parts are easy to use, and the stability is good. It is beneficial to ensure the stability and reliability of the product performance.

### BRIEF DESCRIPTION OF THE DRAWINGS

FIG. 1 is a perspective view according to a preferred embodiment of the present invention;

FIG. 2 is an exploded view according to the preferred embodiment of the present invention;

FIG. 3 is a cross-sectional view according to the preferred embodiment of the present invention;

FIG. 4 is an enlarged view of circle A of FIG. 3; and

FIG. 5 is an exploded view of the ultrasonic transducer according to the preferred embodiment of the present invention, showing the cleaning head.

### DETAILED DESCRIPTION OF THE PREFERRED EMBODIMENTS

Embodiments of the present invention will now be described, by way of example only, with reference to the accompanying drawings.

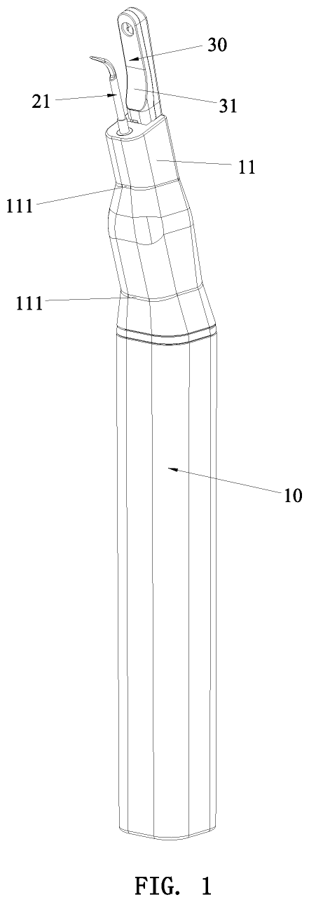

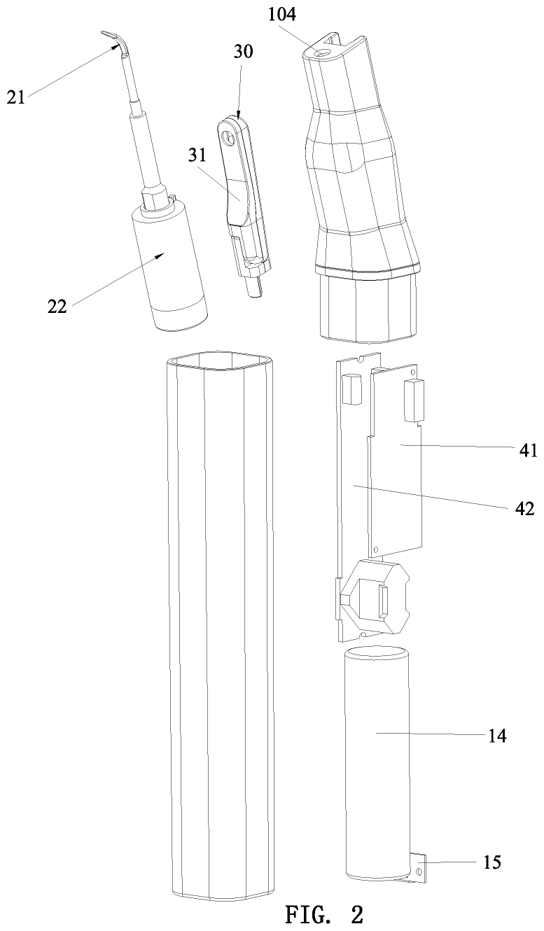

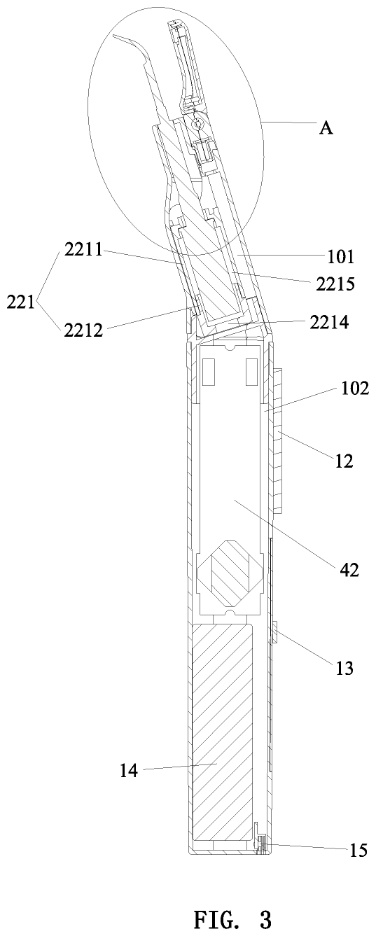

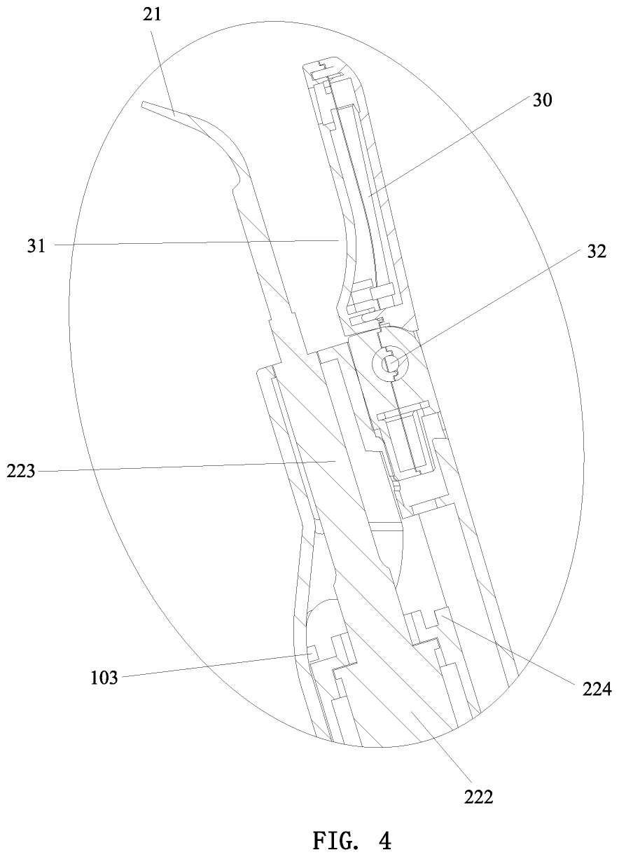

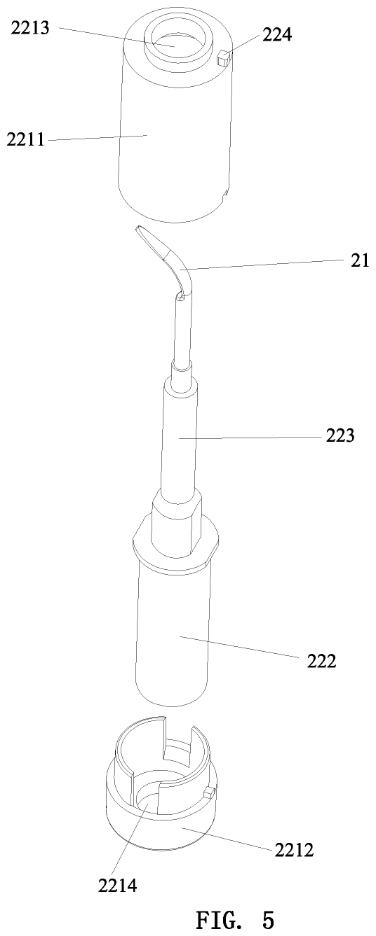

As shown in FIGS. 1 to 5, a tooth cleaning device comprises a main body 10, a cleaning head 21 extending out of the main body 10, and a camera unit 30. The camera unit 30 is arranged toward the cleaning head 21.

An upper end of the main body 10 is provided with an inclined portion 11 that is inclined downward and rearward. The camera unit 30 is mounted to the inclined portion 11. A front end face of the camera unit 30 is formed with a first finger recess 31 corresponding to the curvature of a user's finger surface. Through the first finger recess 31, the user's finger can be positioned when turning the camera unit 30. The anti-slip effect is better. A front end face of the inclined portion 11 is formed with a second finger recess 111 corresponding to the curvature of a user's finger surface. The second finger recess 111 facilitates the use of this product, thereby avoiding slippage and shifting when using this product. The anti-slip effect is better.

An ultrasonic transducer 22 is mounted to the inclined portion 11. The cleaning head 21 is connected to the ultrasonic transducer 22. The ultrasonic transducer 22 is provided with a rotation limiting portion 224 for limiting the rotation angle of the ultrasonic transducer 22. The rotation limiting portion 224 protrudes from an upper end face of the ultrasonic transducer 22, and correspondingly, a locking protrusion 103 is provided on the inclined portion 11. Preferably, the locking protrusion 103 is formed on the inner wall of a first mounting cavity described below, and the rotation limiting portion 224 abuts against the locking protrusion 103.

Preferably, the ultrasonic transducer 22 includes a mounting seat 221, a cylindrical portion 222 mounted to the mounting seat 221, and a driving portion 223 integrally connected to a top end of the cylindrical portion 222. In this embodiment, the driving portion 223 is integrally connected to the cleaning head 21. Of course, the driving portion 223 may be detachably connected to the cleaning head 21, but not limited thereto. The rotation limiting portion 224 is disposed on the mounting seat 221 and/or the cylindrical portion 222. Preferably, the rotation limiting portion 224 is disposed on the upper end face of the mounting seat 221.

The mounting seat 221 includes a seat body 2211 and a bottom cover 2212 that are detachably connected. The rotation limiting portion 224 is formed on the upper end face of the seat body 2211. Of course, the rotation limiting portion 224 may be formed on the peripheral side of the seat body 2211, but not limited thereto. The seat body 2211 and the bottom cover 2212 are assembled with each other to form an inner cavity 2215. The seat body 2211 is formed with a first receiving hole 2213. The bottom cover 2212 is formed a second receiving hole 2214. Both the first receiving hole 2213 and the second receiving hole 2214 communicate with the inner cavity 2215. The cylindrical portion 222 is installed in the inner cavity 2215. The driving portion 223 extends out of the mounting seat 221 from the first receiving hole 2213.

The ultrasonic transducer 22 is rotatably connected to the main body 10. The camera unit 30 is connected to the inclined portion 11 and adjustable to avoid the cleaning head 21 when rotating.

In this embodiment, the camera unit 30 is rotatably connected to the main body 10 to be turned back and forth along its axial direction. For example, the camera unit 30 is pivotally connected to the main body 10 through a pivot shaft 32. When the cleaning head 21 rotates, the camera unit 30 can be turned back to avoid the cleaning head 21.

It should be noted that the camera unit 30 is connected with a wireless communication module 41 for wireless communication with a display device; or, the camera unit 30 is connected with a connection line or a connection port for wired connection with the display device. In this embodiment, the camera unit 30 is connected to a mobile terminal through the wireless communication module 41, so that the operator can clearly observe the cleaning part of his/her teeth through the mobile terminal, and can upload the captured pictures to the Internet to realize remote diagnosis and treatment of patients by dentists or health experts.

In this embodiment, a display screen 12 and a function button 13 are provided on the main body 10. The working status of the main body 10 can be understood in real time through the display screen 12. A power source 14 for power supply is provided in the main body 10. A charging port 15 is provided on a lower end face of the main body 10. The charging port 15 is electrically connected to the power source 14.

In this embodiment, an upper first mounting cavity 101 and a lower second mounting cavity 102 communicating with each other are formed in the main body 10. The top end of the first mounting cavity 101 has an upper through hole 104. The ultrasonic transducer 22 is mounted in the first mounting cavity 101, and the cleaning head 21 extends out of the upper through hole 104. The wireless communication module 41 and the power supply 14 are both mounted in the second mounting cavity 102. A circuit board 42 is also mounted in the second mounting cavity 102. The circuit board 42 is electrically connected to the ultrasonic transducer 22, the wireless communication module 41, the display screen 12 and the function buttons 13 respectively.

What is claimed is:

1. A tooth cleaning device, comprising a main body, a cleaning head extending out of the main body, and a camera unit;
an upper end of the main body being provided with an inclined portion that is inclined downward and rearward, the camera unit being mounted to the inclined portion, an ultrasonic transducer being mounted to the inclined portion, the cleaning head being connected to the ultrasonic transducer, the ultrasonic transducer being rotatably connected to the main body, the camera unit being connected to the inclined portion and adjustable to avoid the cleaning head when rotating.

2. The tooth cleaning device as claimed in claim 1, wherein the ultrasonic transducer is provided with a rotation limiting portion for limiting a rotation angle of the ultrasonic transducer.

3. The tooth cleaning device as claimed in claim 2, wherein the rotation limiting portion protrudes from an upper end face of the ultrasonic transducer, a locking protrusion is provided on the inclined portion, and the rotation limiting portion abuts against the locking protrusion.

4. The tooth cleaning device as claimed in claim 2, wherein the ultrasonic transducer includes a mounting seat, a cylindrical portion mounted to the mounting seat, and a driving portion integrally connected to a top end of the cylindrical portion; the driving portion is detachably connected to the cleaning head, and the rotation limiting portion is disposed on the mounting seat and/or the cylindrical portion.

5. The tooth cleaning device as claimed in claim 1, wherein a function button is provided on the main body.

6. The tooth cleaning device as claimed in claim 1, wherein a display screen is provided on the main body.

7. The tooth cleaning device as claimed in claim 1, wherein the camera unit is rotatably connected to the main body to be turned back and forth.

8. The tooth cleaning device as claimed in claim 1, wherein a front end face of the camera unit is formed with a first finger recess corresponding to the curvature of a user's finger surface.

9. The tooth cleaning device as claimed in claim 1, wherein a front end face of the inclined portion is formed with a second finger recess corresponding to the curvature of a user's finger surface.

10. The tooth cleaning device as claimed in claim 1, wherein a power source for power supply is provided in the main body, a charging port is provided on a lower end face of the main body, and the charging port is electrically connected to the power source.
